(19) United States
(12) Patent Application Publication
(10) Pub. No.: US 2004/0048318 A1
(43) Pub. Date: Mar. 11, 2004

(54) Method for screening anti-proliferative compounds and method for inhibiting tumor growth

(76) Inventors: Penny Elizabeth Phillips, Morristown, NJ (US); Patrick Andre Schindler, Muespach (FR); Harry Towbin, Allschwil (CH)

Correspondence Address: THOMAS HOXIE, NOVARTIS, CORPORATE INTELLECTUAL PROPERTY, ONE HEALTH PLAZA 430/2, EAST HANOVER, NJ, 07936-1080, US

(21) Appl. No.: 10/416,669

(22) PCT Filed: Dec. 11, 2001
(86) PCT No.: PCT/EP01/13076

(51) Int. Cl. G01N 33/574

(52) U.S. Cl. 435/7.23

(57) ABSTRACT

The invention relates to a novel method for evaluating the antiproliferative activity of compounds having MetAP inhibitory activity, as well as a method for screening compounds that inhibit angiogenesis or growth of tumors. The invention additionally provides a method for monitoring the progress of treatment for controlling angiogenesis or the growth of tumors.

The inv ntion provides compositions and methods for treating or preventing neoplasia in human and veterinary patients, compositions and methods for screening a library of agents for pharmacological activity in regulating cell proliferation and/or cell differentiation, compositions and methods for modulation of a transformed cell phenotype in vitro, including use in bioprocess control and as commercial laboratory reagents. This invention further relates to a novel method for evaluating the antiproliferative activity of compounds having MetAP inhibitory activity.

In all living cells, protein synthesis is Initiated with an AUG codon. This initiation AUG codon specifies methionine. The NH₂-methionine, in both eukaryotes and prokaryotes, is removed by methionine aminopeptidases (MetAPs), if the penultimate residue is small and uncharged, e.g. Ala, Cys, Gly, Pro, Ser, Thr and Val. Removal of the NH₂-methionine is essential for certain proteins to function normally in vivo. These proteins can be categorized into at least two groups. For the first group, the removal of the initiator methionine is required for subsequent N-terminal modifications, such as N-myristoylation, which is essential for their normal function, including signal transduction, certain cancer cell growth and protein targeting. For the second group, the removal of the initiator methionine is required to allow the other N-terminal residues to function normally in their critical roles in catalysis. Some proteins when overexpressed in bacteria or other organisms, in which limited MetAPs are available, may still have the undesired initiator methionine attached to their N-termini, and thus become nonfunctional. Accordingly, regulating MetAPs may be a useful method for treating proliferative diseases, e.g., cancer, and other human diseases. In fact, regulation of MetAP, for example with fumagillin, has been shown to be a promising method for treating cancer. Fumagillin appears to inhibit one of two known MetAPs, i.e., MetAP2, by irreversibly blocking its active site.

A family of proteins known as 14-3-3 proteins was first identified by Moore, B. F. et al. (1967) as very abundant 27-30 kD acidic proteins of brain tissue (Physiological and Biochemical Aspects of Nervous Integration, F. D. Carlson, Ed., Prentice-Hall, Englewood Cliffs, N.J., 1967). Their name reflects the original investigators' nomenclature. Recent work has Implicated the participation of 14-3-3 proteins in cell cycle control (Ford, J. C. et al. (1994) Science 265:533). (For a general review, see Morrison, D. (1994) Science 266:56-57 and Aitken, A. (1995) TIBS 20). A variety of functions have been ascribed to the 14-3-3 proteins. However, several lines of evidence suggest that they link signal transduction cascades with the cell cycle. Various 14-3-3 isotypes have been found in complexes with proteins that transform cells, e.g., the middle T antigen of polyoma virus, Bcr-Ab1 and PI3k, with signalling molecules, e.g., c-Raf 1, c-Bcr and P13k, and with two cell cycle regulators, e.g., Cdc25A and CdcB.

Although a limited number of compounds that regulate MetAP activities are known, there remains a need for identifying additional active compounds that regulate MetAP activities.

The structure of the active agents identified herein by code nos., generic or trade names may be taken from the actual edition of the standard compendium “The Merck Index” or from databases, e.g. Patents International (e.g. IMS World Publications). The corresponding content thereof is hereby incorporated by reference.

### SUMMARY OF THE INVENTION

The invention relates to a novel method for evaluating the anti-proliferative activity of compounds having MetAP inhibitory activity. The present invention provides a method for screening compounds that inhibit growth of tumors. The invention additionally provides a method for monitoring the progress of treatment for controlling proliferative diseases. Once compounds with a high level of anti-proliferative activity are identified, these compounds can be used as anti-cancer agents in mammals, including animals in veterinary medicine, or humans in need of treatment for cancer, as well as other proliferative diseases.

It has been found that a 14-3-3γ isoform, more specifically an altered 14-3-3γ isoform, is induced when a MetAP inhibitor or modulator compound is applied to mammalian cells, and the induced protein can be used as a marker for monitoring efficacy of the applied compound. The induced protein can be used as a marker for screening and identifying new compounds that are MetAp inhibitors, and for monitoring systemic efficacy of a selected active compound. For example, the protein marker can be used to screen compounds that provide anti-angiogensis or anti-tumor activities. A range of available cell lines can be treated with an active compound and tested for elevated expression of the altered 14-3-3γ isoform. For example, when U20S cells are treated with bengamide or fumagillin, the cells express the altered 14-3-3γ isoform.

Additionally, the protein marker can be employed to monitor the progress of treatment of a subject with an active compound. An active compound is administered to a subject, e.g., a human patient, and the altered 14-3-3γ isoform level of a test specimen, e.g., white blood cells, from the subject is periodically checked for the altered level or induction of the protein marker. When an active compound having the desired effect (e.g., anti-angiogenesis activities) is administered to a subject, the altered 14-3-3γ isoform level is elevated in the system of the subject.

### DETAILED DESCRIPTION OF THE INVENTION

The present invention provides a method for screening compounds that inhibit or modulate enzymatic activities of MetAPs. It has been found that when the enzymatic activity of MetAPs is interrupted, an altered 14-3-3γ isoform is induced and accumulates in the cell. The altered 14-3-3γ isoform is characterized in that the isoform is an incompletely processed form, having an N-terminal methionine in place of the acetyl group at the N-terminus of the constitutive 14-3-3γ isoform. That is, the N-terminus of the altered human 14-3-3γ isoform has the last seven amino acid sequence of MetValAspArgGluGlnLeu, in place of acetyl-ValAspArgGluGlnLeu of the constitutive 14-3-3γ isoform. It is to be noted that the length of the illustrated amino acid sequence is selected as an example.

Levels of expression of the altered 14-3-3γ isoform (methionine 14-3-3γ) can be assayed from a biological sample, e.g., cell lysate, tissue lysate or white blood cell lysate, by any known methods, including immunoassays and electrophoresis assays. For example, immunoassays can be used to detect or monitor induction and accumulation of methionine 14-3-3γ in a biological sample. Methionine 14-3-3γ-specific polyclonal or monoclonal antibodies can be used in any standard immunoassay format to measure the methionine 14-3-3γ level. ELISA (enzyme linked immunosorbent assay) type assays and conventional Western blotting assays using monoclonal antibodies are exemplary assays that can be utilized to make direct determination of the induction and accumulation of the marker protein. Antibodies specific to methionine 14-3-3γ can be produced in accordance with known immunization methods. For example, monoclonal antibodies against methionine 14-3-3γ are produced by a conventional technique using the aminoterminal peptide sequence of the above-indicated human origin coupled to keyhole limpet hemocyanin as an immunogen.

Another method that can be used to determine the methionine 14-3-3γ level is two-dimensional gel electrophoresis. Two-dimensional gel electrophoresis is known in the art and typically involves isoelectric focusing (IEF) along a first dimension followed by SDS-PAGE (sodium dodecyl sulphate-polyacrylamide gel electrophoresis) along a second dimension. The resulting electropherograms can be analyzed, for example, by immurioblot analysis using antibodies. Suitable antibodies can be produced as discussed above or obtained from a commercial source. For the immunoblotting analysis, the antibody does not have to be specific to methionine 14-3-3γ, but it can be antibodies that are reactive to 14-3-3 or 14-3-3γ in general, i.e., it is reactive to all isotypes of 14-3-3 or specific to the two isoforms of 14-3-3γ having acetylated and methionine N-termini, respectively. Using IEF, methionine 14-3-3γ and constitutive acetylated 14-3-3γ can be easily separated. The methionine of the induced 14-3-3γ isoform, which has the methionine terminal residue, is positively charged at neutral pH, and thus, the methionine 14-3-3γ isoform is more positively charged than the constitutive acetyl 14-3-3γ isoform, allowing the two isoforms to be separated by pl.

Various compounds that inhibit or modulate the enzymatic activities of MetAPs (MetAP inhibitors) include fumagillin, ovalicin, benegamide, and certain analogues and derivatives thereof. Fumagillin, ovalicin, and analogues and derivatives thereof are known to inhibit the MetAP2 enzymatic activities, while it is believed that bengamide and analogues and derivatives thereof inhibit both MetAP1 and MetAP2. The MetAP inhibitors are highly useful as a cytotoxic agent for treating proliferative diseases including tumors, e.g., cancer. MetAP inhibitors are particularly suitable for inhibiting the growth of various lymphomas, sarcomas, carcinomas and myelomas. In addition, MetAP inhibitors are suitable for treating angiogenesis-dependent diseases, e.g., various ocular neovascular diseases.

The present invention can be utilized to Identify other compounds that have MetAP inhibition activities. Various in vitro and in vivo experiments can be employed to screen potential compounds. For example, a cell culture is treated with a compound and cultivated, then the culture is assayed to determine the expressed level of methionine 14-3-3γ. Induction of methionine 14-3-3γ in the cell culture indicates that the compound is a MetAP inhibitor. It has been found that accumulation of methionine 14-3-3γ in cells is not a cell cycle dependent event, but it is induced by a MetAP inhibitor. Again, detection and quantification of methionine 14-3-3γ in a biological sample can be conducted by various methods, as discussed above.

The present invention also provides a method for monitoring therapeutic efficacy of an active compound, which inhibits or regulates MetAP activities. The present invention can be used as a clinical marker to monitor efficacy of a MetAP inhibitor compound on each patient. When a MetAP inhibitor compound is therapeutically administered to a patient, an elevated level of methionine 14-3-3γ is observed in a biological sample, e.g. blood or tissue, especially in the target cells, from said patient when the compound has a beneficial effect on the proliferative condition of the patient. Similarly, the clinical marker can be used to optimize the dosage and the regimen of an active compound by monitoring the induction and accumulation of methionine 14-3-3γ in the subject's biological sample. Accordingly, the screening method of the present invention can be used to find a therapeutically effective compound and/or to find a therapeutically effective amount or regimen for the selected compound, thereby individually selecting and optimizing a therapy for a patient. Factors for consideration in this context include the particular condition being treated, the particular mammal being treated, the clinical condition of the individual patient, the site of delivery of the active compound, the particular type of the active compound, the method of administration, the scheduling of administration, and other factors known to medical practitioners. The therapeutically effective amount of an active compound to be administered will be governed by such considerations, and is the minimum amount necessary to prevent, ameliorate, or treat the disease. Such amount is preferably below the amount that is toxic to the host or renders the host significantly more susceptible to infections.

In particular, the present invention provides a method for screening a therapeutic compound for an antiproliferative indication, comprising administering said compound to a mammalian subject and testing for methionine 14-3-3γ in a biological sample from said mammalian subject.

Similarly, the present invention provides a method for screening a therapeutic compound for an antiproliferative indication, comprising testing for methionine 14-3-3γ in a biological sample, said biological sample having been obtained from a mammalian subject to which said therapeutic compound had been administered.

The present invention also provides the use of a test for detecting methionine 14-3-3γ in a biological sample to screen a therapeutic compound for an antiproliferative indication, said biological sample having been obtained from a mammalian subject to which said therapeutic compound had been administered.

In a further embodiment, the present invention provides a method for screening a compound having remedial effect on a cellular proliferative condition, comprising adding said compound to a cellular system and testing for induction of methionine 14-3-3γ in said system.

The present invention also provides the use of a test for detecting induction of methionine 14-3-3γ in a cellular system to screen a compound which has a remedial effect on a cellular proliferative condition, whereas said compound is added to said cellular system before detection of methionine 14-3-3γ.

In yet another embodiment, the present invention provides a method for screening a compound having MetAP Inhibitory activities, comprising adding said compound to a cellular system and testing for an altered expression of 14-3-3γ in said system.

The present invention also provides the use of a test for detecting an altered expression of 14-3-3γ in a cellular system to screen a compound which has MetAP inhibitory activities, whereas said compound is added to said cellular system before detection of 14-3-3γ.

The present invention further provides a method for inhibiting MetAP activity in a mammal, comprising administering to said mammal a compound having an ability to Induce methionine 14-3-3γ, said compound being administered in an amount sufficient to induce said 14-3-3γ in the mammal.

Similarly, the present Invention provides a method of inhibiting cell proliferation in tumor tissue or the peripheral blood lymphocyte in a mammal, comprising administering a compound having an ability to elevate methionine 14-3-3γ levels in said proliferative tissue or lymphocyte, in an amount sufficient to elevate said methionine 14-3-3γ levels.

The present invention further provides a method of identifying antiproliferative activity of compounds having MetAP inhibitory activity, comprising the steps of

a) preparing a proliferative cell culture,

b) inoculating said cell culture with a compound having MetAP inhibitory activity in an amount effective for Increasing the level of methionine 14-3-3γ in the cell culture,

c) and isolating cells from said cell culture and measuring the level of methionine 14-3-3γ produced in the cell culture.

In a preferred embodiment, the present invention provides a method of the preceding paragraph in which the proliferative cell culture is a small cell lung carcinoma, a non-small cell lung carcinoma, an osteosarcoma, a human breast carcinoma, or a contact inhibited mouse fibroblast cell line.

In a further preferred embodiment, the present invention provides a method of the preceding penultimate paragraph in which the compound having MetAP inhibitory activity is selected from the group consisting of fumagillins, ovalicins, bengamids, and caprolactams, including LAF389 and TNP-470.

The following examples further illustrate the present invention, and the examples are provided for illustration purposes and are not intended to be limiting the invention thereto.

### EXAMPLE 1

H1299, a non-small cell lung carcinoma which is obtained from the American Type Culture Collection (Rockville, Md.), is cultivated in PRMI1640 supplemented with 10% fetal calf serum and glutamine, penicillin, streptomycin, and gentamycin. The H1299 cells are grown to about 70% confluenc and then cultivated with or without 25 μM bengamide. Samples of the cells are taken at 8, 16, 24 and 48 hours. At the end of the culture periods, the sample cells are washed with PBS (phosphate buffered solution, available from Sigma Chemical) and collected by scraping and centrifugation. The cell pellet is dissolved in 4 volumes of Rabilloud buffer. Rabilloud buffer contained 4% non-ionic detergent CHAPS, 7 M urea, 2 M thiourea, 10 mg/ml dithiothreitol and 1% pharmalytes (pH 3-10), which buffer is disclosed in Electrophoresis, 1997 Mar-Apr., 18 (3-4): 307-16, Rabilloud et al., Improvement of the Solubilization of Proteins in Two-dimensional electrophoresis with immobilized pH Gradients. The cell concentration was between 2 and 2.5 E7/ml.

Immobilized pH-gradient (IPG) gels (Immobiline®, Amersham Pharmacia Biotech) are re-swollen for at least 4 hours in Rabilloud buffer without pharmalytes. Samples (1 μl of cell extract) are directly loaded at a distance of about 2 cm from the anode to the surface of the gel using a comb. Focusing is carried out at 200 V for 2 hours and at 1000 or 3500 V until a 25000 Vh is reached. The temperature is maintained at 15° C. After focusing, the gel is fixed in 12% TCA-3.5% sulfosalicylic acid for 1 hour and washed in water for 1 hour. The washed gel is blotted by placing the gel on a flat surface and covering with a sheet of PVDF-membrane which was sequentially soaked in Isopropanol, water and a guanidinium chloride solution of 50 nM Tris-HCl at pH 7.5, 4 M guanidinium chloride, 1 mg/ml DTT. The membrane is covered with 4 layers of filter paper sheets which was soaked with the guanidinium chloride solution. A polyethylene film is placed over the filter sheets and a 3 kg weight having a 14 cm by 14 cm flat surface is placed over the polyethylene film to ensure a good contact between the layers. The gel layers are undisturbed to allow transfer for overnight, then the membrane is washed with water and treated with a solution of 0.5% formaldehyde in PBS for 30 minutes. The membrane is incubated with 2% glycine in PBS at pH 7 for 30 minutes.

The membrane is then blocked with 0.05% Tween 20-1% skimmed dry milk powder in PBS for 1 hour. Commercially available antibodies against 14-3-3 (Santa Cruz) are applied for 4 hours in blocking buffer. Chemiluminescence detection (ECL-kit, Amersham Pharmacia Biotech) is carried out using the secondary antibodies supplied with the kit.

The IEF-immunoblotting clearly Indicates that the cells cultured with bengamide gradually accumulate over the 48 hour culture period the protein that reacts with the 14-3-3γ specific antibody and that corresponded to the IEF position of methionine 14-3-3γ, whereas the cells cultured without bengamide do not produce any noticeable amount of the methionine 14-3-3γ protein over the 48 hour period. The IEF-immunoblotting result clearly demonstrates that protein reactive to the 14-3-3γ antibody is induced by bengamide.

### EXAMPLE 2

Example 1 is repeated except that 200 nM of the bengamide analogue LAF389 having the following formula

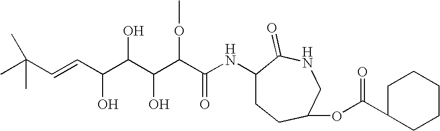

is used in place of bengamide.

The IEF-immunoblotting result is similar to that of Example 1. The cells cultured with the bengamide analogue induce and accumulate the protein which is reactive to the 14-3-3γ antibody, whereas the cells cultured without the bengamide analogue do not induce methionine 14-3-3γ.

### EXAMPLE 3

Example 1 is repeated with a known MetAP2 inhibitor, a fumagillin compound. An analogue of fumagillin, AGM-1470 (TNP-470), is used in place of bengamide. The IEF-immunoblotting analysis demonstrates that the cells cultured with AGM-1470 induce methionine 14-3-3γ while the untreated cells do not induce appreciable amounts of the inducible protein.

### EXAMPLE 4

Example 2 is repeated with the following cell lines, which are obtained from the American Type Culture Collection, Rockville, Md.: MDA-MB435 (mammary carcinoma), A549 (lung carcinoma) and U20S (osteosarcoma). The IEF-immunoblotting analysis demonstrates that all of the tested cell lines induce methionine 14-3-3γ within 18 hours after the MetAP Inhibitor is administered.

### EXAMPLE 5

One group of laboratory rats is treated with the bengamide analogue LAF389 of Example 2 three times for a day at the 3 mg/kg dosage, then rested for 7 days. Th rats are again dosed three times the following day with the dosage. They are rested for one day and blood samples are collected. Peripheral blood is collected by a percoll gradient centrifugation protocol. The white blood cell fraction including platelets is recovered and the pellet is lysed in IEF sample buffer. The bio-samples are analyzed with the IEF-immunoblotting analysis. Control bio-samples are obtained from a group of rats that are not treated with the bengamide analogue.

The IEF-immunoblotting result demonstrates that the rats treated with the MetAP inhibitor induce and accumulate methionine 14-3-3γ while the control rats do not induce the marker protein, clearly demonstrating in vivo efficacy of a MetAP inhibitor in a mammal.

### EXAMPLE 6

Various antineoplastic compounds that are known to be non-MetAP inhibitors are used on the H1299 cells to test for the induction of methionine 14-3-3γ. The following compounds are applied on the cells: 5-fluorouracil (20 μg/ml), epothilone (500 nM), doxorubicin (1 μg/ml) and stauosporin (1 μM), along with 200 nM of the bengamide analogue LAF389. The cells are incubated for 18 hours and tested for methionine 14-3-3γ by following the procedure outlined in Example 1.

The IEF-immunoblotting analysis shows that only the MetAP inhibitor induced methionine 14-3-3γ. The result demonstrates that the antineoplastic compounds which are not MetAP inhibitors do not induce methionine 14-3-3γ.

### EXAMPLE7

Monoclonal Antibodies (mAbs) against the amino terminus of 14-3-3γ are raised by conventional techniques using the aminoterminal peptide sequence of human 14-3-3γ coupled to keyhole limpet hemocyanin as an immunogen. Two synthesized peptide sequences are used: MetValAspArgGluGlnLeu, representing the amino terminus of methionine 14-3-3γ, and Acetyl-ValAspArgGluGlnLeu, representing the amino terminus of constitutive 14-3-3γ. The mAbs are isolated in accordance with known methods. Specificity of the mAbs is checked against the peptides used for immunization. The mAbs are used in various assays including ELISA and Western blotting.

What is claimed is:

1. A method for screening a therapeutic compound for an antiproliferative indication, comprising administering said compound to a mammalian subject and testing for methionine 14-3-3γ in a biological sample from said mammalian subject.

2. A method for screening a compound having remedial effect on a cellular proliferative condition, comprising adding said compound to a cellular system and testing for induction of methionine 14-3-3γ in said system.

3. A method for screening a compound having MetAP inhibitory activities, comprising adding said compound to a cellular system and testing for an altered expression of 14-3-3γ in said system.

4. A method for inhibiting MetAP activity in a mammal, comprising administering to said mammal a compound having an ability to induce methionine 14-3-3γ, said compound being administered in an amount sufficient to induce said 14-3-3γ in the mammal.

5. A method of inhibiting cell proliferation in tumor tissue or the peripheral blood lymphocyte in a mammal, comprising administering a compound having an ability to elevate methionine 14-3-3γ levels in said proliferative tissue or lymphocyte, in an amount sufficient to elevate said methionine 14-3-3γ levels.

6. A method of identifying antiproliferative activity of compounds having MetAP inhibitory activity, comprising the steps of
a) preparing a proliferative cell culture,
b) inoculating said cell culture with a compound having MetAP inhibitory activity in an amount effective for increasing the level of methionine 14-3-3γ in the cell culture,
c) and isolating cells from said cell culture and measuring the level of methionine 14-3-3γ produced in the cell culture.

7. The method of claim 6 in which the proliferative cell culture is a small cell lung carcinoma, a non-small cell lung carcinoma, an osteosarcoma, a human breast carcinoma, or a contact inhibited mouse fibroblast cell line.

8. The method of claim 6 in which the compound having MetAP inhibitory activity Is selected from the group consisting of fumagillins, ovalicins, bengamids, and caprolactams, including LAF389 and TNP-470.
